(19) United States
(12) Patent Application Publication
(10) Pub. No.: US 2004/0253660 A1
(43) Pub. Date: Dec. 16, 2004

(54) Automated microbiological testing apparatus and method

(75) Inventors: David Gibbs, Santa Barbara, CA (US); Qi (Andrew) Wang, Santa Barbara, CA (US)

Correspondence Address: R. Neil Sudol, 714 Colorado Avenue, Bridgeport, CT, 06605-1601, US

(73) Assignee: GILES SCIENTIFIC, INC., Santa Barbara, CA

(21) Appl. No.: 10/460,708

(22) Filed: Jun. 12, 2003

(51) Int. Cl. C12Q 1/04; G06F 19/00; G01N 33/48; G01N 33/50

(52) U.S. Cl. 435/34; 702/19

(57) ABSTRACT

A microbiological testing method or assay for identifying an organism grown on one chromagenic semisolid nutrient media such as agar, where the organism exhibits at least one color or chromatic aspect. A digitized electrical signal is generated encoding an image of the organism on the nutrient media. The encoded image is stored and digitally processed to detect the color of the organism on the nutrient media. Chromatic characteristics of a multiplicity of known organisms are stored in an electronic library. A computer is operated to compare the detected color of the organism with chromatic characteristics stored in the library.

### BACKGROUND OF THE INVENTION

The present invention relates to a microbiological testing apparatus and an associated method. More specifically, the present invention relates to an apparatus and associated method for use in identification and optionally colony counting and or antibiotic susceptibility testing of samples, such as those from patients possibly infected by a microorganism.

A growing portion of microbiological sample testing currently performed relies on the visual inspection of chromagenic nutrient plates, commonly known as chrome agar, to identify microbiological species by the colors of colonies appearing on the chromagenic nutrient media. This method is expensive and time consuming. Personnel must be trained to distinguish closely related colors. In addition, the visual monitoring of the chromagenic plates takes considerable time even for experienced personnel.

Many automated systems have been proposed and/or developed for reading results of microbiological test samples for providing information on the identification of the causative organism, and the susceptibility of that organism to various antimicrobial agents. Other automated systems have been proposed and/or developed to count organism colonies. Several proposed or existing automated systems include moving of sample-containing assay trays and sensors relative to one another. Of course, such systems, with their motors and moving parts, require costly and complex servicing. Accordingly, it is not only desirable to arrive at test results very rapidly, but it is also advantageous to eliminate motors and minimize moving parts that require costly and complex servicing.

Existing systems usually require that an organism be grown as one isolated organism from a patient's specimen, and that a specific large number of organisms of this isolate compose an inoculum for various separate biochemical tests for identification and separately for antibiotics testing that requires further incubation time. This process is slow, cumbersome, and costly.

### OBJECTS OF THE INVENTION

A general object of the present invention is to provide an apparatus and/or associated method for microbiological testing.

A more specific object of the present invention is to provide a microbiological-testing apparatus and/or method for use in identifying species by color produced by colonies on chromogenic semisolid/agar nutrient media.

Another object of the present invention is to provide an essentially automated microbiological testing apparatus and/or method wherein color detecting and evaluation by image analysis is performed automatically.

A further object of the present invention is to provide such an automated microbiological testing apparatus and/or method wherein motors and other moving parts are minimized, if not eliminated.

Another object of the present invention is to provide such an automated microbiological testing apparatus and/or method wherein an organism may be identified using only one plate of chromogenic media.

Another object of the present invention is to provide such an automated microbiological testing apparatus and/or method wherein several species of organisms may be identified using only one plate of chromogenic media.

Another object of the present invention is to provide such an automated microbiological testing apparatus and/or method wherein an organism may be identified from the primary or initial agar media plate on which it was first grown in order to reduce the time to identification.

Another object of the present invention is to provide such an automated microbiological testing apparatus and/or method whereby, with some organisms, an antibiotic susceptibility disk test may be read simultaneously on the same nutrient media as the identification.

Another object of the present invention is to provide such an automated microbiological testing apparatus and/or method wherein, in some cases, the number of colonies of organisms may be counted simultaneously on the same nutrient agar plate with the identification.

An additional object of the present invention is to provide such an automated microbiological testing apparatus and/or method wherein test results are determined based on as much informational experience as possible. These and other objects of the present invention will be apparent from the descriptions and illustrations provided herein. While every object of the invention is believed to be attained by at least one embodiment of the invention, there is not necessarily any one embodiment that achieves all objects of the invention.

### SUMMARY OF THE INVENTION

The present invention is directed in part to a microbiological testing method or assay for identifying an organism grown in at least one colony on semisolid chromagenic nutrient media such as agar, where the organism exhibits at least one color or chromatic aspect. The method comprises generating a digitized electrical signal encoding an image of the colony of organism(s) and surrounding media on the nutrient media, storing the encoded image, digitally processing the encoded image to detect the color(s) of the organism(s) on the nutrient media, storing chromatic characteristics of a multiplicity of known organisms in an electronic library, and operating a computer to compare the detected color of the organism with chromatic characteristics stored in the library.

The operating of the computer preferably includes calculating, for each given one of a plurality of known organisms with pre-identified chromatic characteristics stored in encoded form in the library, a probability that the organism is of the same type as the given one of the known organisms. It is contemplated that the chromatic characteristics stored in the library include, for each of the known organisms, a plurality of chromatic parameters including, for instance, at least one characteristic hue, at least one characteristic saturation, and at least one characteristic value or intensity. It is additionally contemplated that the chromatic parameters each include an average value and a statistical measure of variation about the average value.

As discussed hereinafter, the method of the present invention contemplates the detection of other visible characteristics of colonies, in addition to color. Those other visible characteristics may also be used in the automatic identification process. To that end, representative values and/or ranges of values of the other visible characteristics are stored in the electronic library for a number of known organisms. For instance, each pertinent nonchromatic characteristic of each known organism represented in the library may include an average value and a statistical measure of deviation about that average value. Measured nonchromatic visible characteristics of an unknown organism may be compared with the respective stored nonchromatic characteristics of known organisms. More specifically, the comparison may entail a computation of the probability that the unknown organism is of the same type as one or more organisms with characteristics stored in the electronic library.

In accordance with another feature of the present invention, the method further comprises preliminarily processing the encoded image to detect colonies of the organism on the nutrient media. The preliminary processing of the encoded image generally includes digitally measuring at least one parameter, such as light intensity, distinguishing the colonies from the nutrient media. The processing of the encoded image to detect the color(s) of the organism is performed with reference to image data pertaining to a selected one of the detected colonies.

Pursuant to another feature of the present invention, the method further comprises storing in the library at least one non-chromatic optical characteristic of each of the known organisms. The digital processing of the encoded image then includes measuring the non-chromatic optical characteristic of the organism on the nutrient media, while the computer is additionally operated to compare the measured non-chromatic optical characteristic of the organism with the non-chromatic optical characteristics stored in the library.

The non-chromatic optical characteristic may be a size or textural characteristic, which is measured by detecting edges in the encoded image. In the case of a textural characteristic, the detected edges per unit area are counted.

Where the library includes chromatic and non-chromatic optical characteristics of known organisms and images are processed to detect color and non-color characteristics of an unidentified organism, the computer is operated to calculate, for each given one of a plurality of known organisms with pre-identified chromatic characteristics and pre-identified non-chromatic optical characteristics stored in encoded form in the library, a probability that the unidentified organism is of the same type as the given one of the known organisms. The chromatic characteristics and the non-chromatic optical characteristics may each include an average value and a statistical measure of variation about the average value.

Pursuant to a further feature of the present invention, the generating of the digitized electrical signal includes scanning the media and the organism with an optical scanner. The optical scanner may be a camera such as a digital camera or a charge-coupled device.

Organisms subject to the present identification methodology are generally yeast, bacteria, or mold.

Where the organism is a mold, the method may further comprise providing the nutrient media with at least one anti-fungal composition, depositing pieces of the mold in an array on the nutrient media provided with the anti-fungal composition, growing the mold on the nutrient media provided with the anti-fungal composition, and measuring effectiveness of the anti-fungal composition. The measuring of effectiveness includes operating the computer to determine a size parameter of mold grown from at least one of the pieces. Microscopic observations and/or the growth rate/incubation duration may be additional information input into the system with some organisms to provide a more specific identification.

A microbiological-assay apparatus comprises, in accordance with the present invention, a support, an optical scanning device, a memory, an electronic library, a digital processor, and a computer. The processor may be realized as a programmed function of the computer. The support serves to hold a container of chromagenic nutrient media wherein an organism of unknown identity is grown, the organism exhibiting at least one color on the nutrient media. The optical scanning device is aimed at the support for generating a digitized electrical signal encoding an image of the organism on the nutrient media. The memory is operatively connected to the scanning device for temporarily storing the encoded image. The digital processor is operatively connected to the memory for analyzing the encoded image to detect the at least one color of the organism on the nutrient media. The electronic library stores chromatic characteristics of a multiplicity of known organisms. The computer is operatively connected to the processor and the library and is programmed to compare the detected color of the organism with chromatic characteristics stored in the library. More particularly, the computer is programmed to calculate, for each given one of a plurality of known organisms with pre-identified chromatic characteristics stored in encoded form in the library, a probability that the organism is of the same type as the given one of the known organisms.

As discussed above, the chromatic characteristics stored in the library may include, for each of the plurality of known organisms, a plurality of chromatic parameters, e.g., a characteristic hue, a characteristic saturation, and a characteristic value or intensity, each encoded as an average value and a statistical measure of variation about the average value.

A preprocessor may be operatively connected to the memory for preliminarily processing the encoded image to detect colonies of the organism on the nutrient media. In particular, the preprocessor may include a module for digitally measuring at least a light intensity parameter. The processor is operatively connected to the preprocessor to operate on image data pertaining to a selected one of the detected colonies.

Where the library stores at least one non-chromatic optical characteristic of each of the known organisms, the processor is adapted to measure the non-chromatic optical characteristic of the organism on the nutrient media, while the computer includes a comparator module for comparing the measured non-chromatic optical characteristic of the organism with the non-chromatic optical characteristics stored in the library. The non-chromatic optical characteristic may comprise a textural characteristic, in which case the processor may include an edge detector and an edge counter. The computer preferably includes a probability calculator for determining, for each given one of a plurality of known organisms with pre-identified chromatic characteristics and pre-identified non-chromatic optical characteristics stored in encoded form in the library, a probability that the organism is of the same type as the given one of the known organisms.

Where the organism is a mold, the nutrient media may be provided with at least one anti-fungal composition, and pieces of the mold are deposited in an array on the nutrient media provided with the anti-fungal composition, the mold being grown on the nutrient media provided with the anti-fungal composition, the computer may include a size detector for determining a size parameter of mold grown from at least one of the pieces to thereby ascertain the susceptibility of the mold to the anti-fungal composition. Microscopic observations and/or the growth rate/incubation duration may be additional information input into the system with some species to provide a more specific identification

A microbiological-assay method for testing an organism grown on semi-solid nutrient media for antibiotic susceptibility comprises, in accordance with another feature of the present invention, utilizes a container of semi-solid nutrient media on which is disposed an organism of unknown type and an elongate strip provided at different locations with different concentrations of an antibiotic composition. After an incubation period, the nutrient media, the strip, and a growth region of the organism are optically scanned by a device such as a digital camera. In response to the optical scanning, a digitized electrical signal encoding an image of the strip and the growth region on the nutrient media is generated. The encoded image is stored and digitally processed to detect an intersection point of an edge of the growth region and the strip. A computer is operated to determine a minimum inhibitory concentration of the antibiotic composition based on the detected intersection point and the category of susceptibility or resistance interpretation.

An automated microbiological testing apparatus in accordance with the present invention operates on one or more of a multiplicity of visible characteristics including color(s) of the organism colony growth, surrounding color of the media, colony size and edges and texture, and other visible characteristics, to make an identification.

In an automated microbiological testing apparatus and method in accordance with the present invention, an organism can be identified by using only one image of one plate of one chromogenic media. In contrast to conventional techniques, it is not necessary to use a multiplicity of plates and/or wells with nutrient media and various biochemicals.

In an automated microbiological testing apparatus and method in accordance with the present invention, it is possible to identify an organism from the primary or initial agar media plate on which a patient's specimen was first grown. This reduces the time to identification, in contrast to conventional techniques that first isolate the organism on the primary media, then sub-culture or transfer it to other media for further testing. The present invention also enables identification of several different species cultured on a single chrome agar plate from a patient's specimen, whereas conventional procedures require a subculturing of various isolates to separate media for further testing.

In an automated microbiological testing apparatus and method in accordance with the present invention, with some organisms, an antibiotic susceptibility disk test may be read simultaneously on the same nutrient media as the identification. This procedure is effectuated using a secondary plate where growth from a primary plate is suspended as an inoculum adjusted quantitatively (to provide a uniform variable required for accurate susceptibility testing). With some organisms this procedure may require the additional input of microscopic observations, and or growth-rate/incubation time. One example of this is the identification and disk susceptibility testing of fungal molds.

In an automated microbiological testing apparatus and method in accordance with the present invention, with some organisms, a count of the colonies of organism(s) may be performed simultaneously on the same nutrient media as the identification. Some of these organisms may require additional input of microscopic observations. One example of this is the simultaneous identification and colony counting of a test of a water sample for bacterial contamination.

### BRIEF DESCRIPTION OF THE DRAWINGS

FIG. 1 is a block diagram of an apparatus in accordance with the present invention, for reading microbe patterns on chromagenic nutrient media or microbiological assay disks.

FIG. 2 is partially a schematic diagram and partially a block diagram, showing another embodiment of an apparatus in accordance with the present invention, for reading microbe patterns on chromagenic nutrient media or microbiological assay disks.

FIG. 3 is a block diagram of a comparator and memory components of the apparatus of FIG. 2.

### DEFINITIONS

The word “colony” is used herein to denote a population of an organism growing on nutrient media such as chrome agar. The organism may be bacteria or yeast, in which case the colony is a collection of substantially identical single-cell organisms. Alternatively, the organism may be a multiple cell organism such as a mold, in which case the colony is a single organism.

The colonies are basically individual organisms that have grown into a clone of offspring. Agar plates containing colonies or interest are usually “primary isolation plates,” that is the infectious specimen from the patient is inoculated directly onto the plate in such a way as to result in the visualization of individual colonies of organisms, that can be selected and used for subsequent identification and antibiotic susceptibility testing. The colonies are detected and tested by reading a combination of various colony properties (color, texture, pattern, size) to identify the bacteria genus or species. Alternatively, the colonies may be taken from a “primary isolation plate” and sub-cultured and grown on a “secondary plate” with a chromogenic media and inoculated to provide clearly isolated colonies of organisms, or a uniform growth of organisms from a qualitatively adjusted inoculum.

The term “color” as used herein is defined as a range of colors with one or more sample colors in the color space, due to the verities of color responses from species on the chrome agar. The term “color” is used generally herein to refer to an RGB (red, green, blue) or an HSI (hue, saturation, intensity) value set.

The term “texture” and related terms as used herein are defined herein as the degree of complexity inside the colony area. This complexity may be characterized, for example, by a sum of the edges in a unit area.

The term “size” as used herein is defined as a range of areas where a specific species falls within a confidence interval (calculated with mean, variance and presumed distribution). Note that for a profile with statistical significance, a relatively large number of known samples must be taken. Generally, size and growth rate based on incubation time are used in the present method as a selection criterion to further process and compare other features.

### DETAILED DESCRIPTION OF THE PREFERRED EMBODIMENT

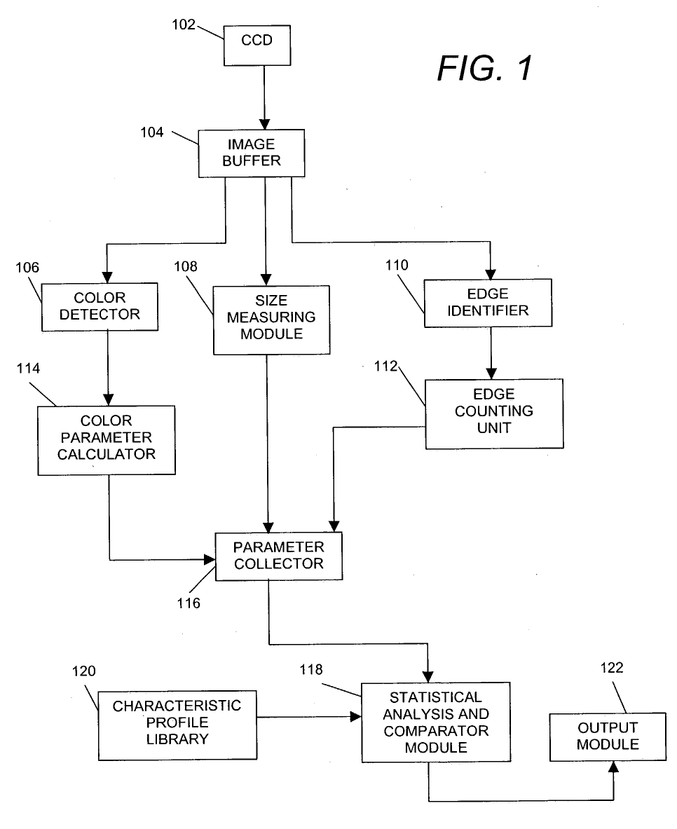

FIG. 1 illustrates a microbiological testing apparatus for use in identifying an organism grown on chromagenic nutrient media such as chrome agar, where the organism exhibits at least one color or chromatic aspect. The microbiological-assay apparatus of FIG. 1 comprises a colorimetric camera 102 in the form of a charge coupled device or CCD adapted for optically scanning a petri dish containing the chrome agar and inoculated with an unknown microbe. CCD 102 generates a digital electrical signal encoding an image of the chrome agar after the microbe has been incubated sufficiently long to result in colonies exhibiting a chromatic aspect.

CCD 102 feeds the digital image signal to an image buffer or memory 104 that in turn provides digitized image data to a color detector 106, a size measuring module 108, and an edge identifier or detector 110. Color detector 106 analyzes the image data stored in buffer 102 to generate colorimetric data associated with each pixel of the image. Size measuring module 108 detects colonies on the nutrient media and measures the size (diameter, radius, area, volume, mass) of those colonies. This measurement, as well as those undertaken by color detector 106 and edge identifier 110, is performed after incubation of the petri dish for a standard time under standard conditions.

Edge identifier or detector 110 cofunctions with an edge counting unit 112 to measure a non-chromatic optical characteristic of the unknown organism, in particular, the texture of the organism's colonies. Edge identifier 110 analyzes the image data in buffer 104 to detect edges in the image. Edge counting unit 112 then tallies the edges detected within various predetermined unit areas of the image.

Color detector 106 is connected at an output to a color parameter calculator 114 that produces colorimetric parameter values of a pre-specified type such as hue, saturation and intensity (HSI) values. Color parameter calculator 114, size measuring module 108, and edge counting unit 112 are connected to a parameter collector 116 that culls the data from those units to determine one or more average color and texture values at locations on the petri dish occupied by grown organisms and to determine the size of the respective colonies. Parameter collector 116 delivers the determined color, size and texture values to a statistical analysis and comparator module 118 that is connected at an input to a characteristic profile library 120 and at an output to a communications interface or output module 122.

Profile library 120 stores chromatic characteristics (HSI values) and associated non-chromatic characteristics such as texture (e.g., edge counts) and, optionally, characteristic colony size (per predetermined growth time) of a multiplicity of known organisms. The color, texture, and size characteristics of the known organisms are each preferably in the form of average values and statistical measures of variation about the respective average values. Library 120 stores, for each known organism, an average hue, an average saturation, and an average intensity, together with respective deviation values an average edge count per unit area and a measure of deviation about that aver edge count, and an average radius, area, volume or mass, as well as a respective deviation value.

For each given known organism with predetermined chromatic characteristics and non-chromatic optical characteristics stored in encoded form in library 120, statistical analysis and comparator module 118 compares color and texture measurements from parameter collector 116 with chromatic and non-chromatic optical characteristics of known organisms stored in library 118. Module 118 supplies the results of its comparisons to communications interface or output module 122. More particularly, module 118 determines a probability that the unknown organism in the chrome agar petri dish is of the same type as the given known organism.

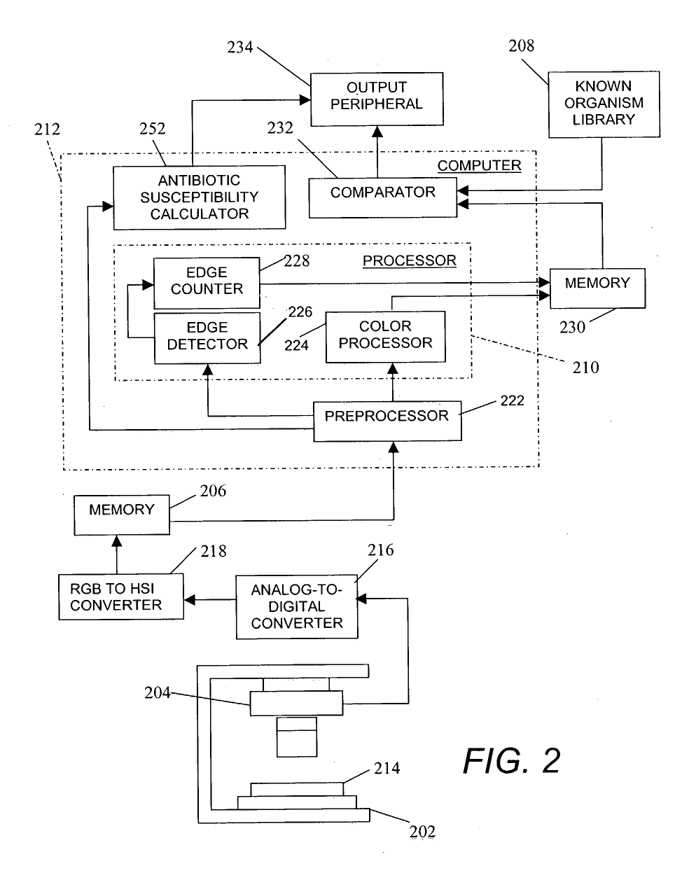

FIG. 2 depicts another, more detailed, embodiment of a microbiological testing apparatus for use in identifying an organism grown on chromagenic nutrient media such as chrome agar, where the organism exhibits at least one color or chromatic aspect. The microbiological-assay apparatus of FIG. 2 comprises a support 202, an optical scanning device 204 in the form of a color camera or charge coupled device (CCD), a memory 206, an electronic profile library 208, a digital processor 210, and a computer 212. Processor 210 is realized as a component of computer 212 and more particularly as generic computer circuits modified by programming to perform functions described below.

Support 202 serves to hold a container or plate 214 (e.g., petri dish) of chrome agar nutrient media wherein an organism of unknown identity is grown, the organism exhibiting at least one color on the nutrient media. Camera or CCD 204 is aimed at support 202 and more specifically at container 214 thereon for generating an electrical signal encoding an image of the organism on the nutrient media. If the camera or CCD 204 produces an analog signal, that signal is digitized by an analog-to-digital converter 216. The digitized image signal is fed to a converter 218 that transforms the signal from RGB (red, green, blue) values to HSI or HSV (hue, saturation, intensity or value) values. The HSI color image signal is temporarily stored in buffer or memory 206.

Buffer or memory 206 is connected at an output to a preprocessor 222. Like processor 210, preprocessor 222 may be realized as a component of computer 212 and more particularly as generic computer circuits modified by programming to perform prescribed functions. Preprocessor 222 preliminarily processes the encoded image from member 220 to detect colonies of the organism on the nutrient media of container 214. In particular, preprocessor 222 may comprise a module (not separately illustrated) for digitally detecting at least a light intensity parameter. Preprocessor or intensity measurement module 222 thus measures light intensity over the area of container 214 to differentiate between colonies and the chrome agar.

Preprocessor 222 implements colony detection by the following specific steps. The color image is converted into a gray scale based on the color of interest specified by the user and the profiles of the media used (blood, clear or paper grid media). Edges are detected and edge pixels are mapped back into the gray image to calculate the threshold. Preprocessor 222 thresholds the image into two levels for colony detection, then searches the detected regions, and marks the bi-level image. Connected regions are merged. An erosion and dilation filter is used to separate touching colonies. Colony properties are calculated, including size, fill factor (ratio of area/size), and color (mapped back into the original color image). Colonies are counted based on the properties.

Preprocessor 222 is connected to a color processor 224 and an edge detector 226 both implemented as parts of processor 210. Color processor 224 operates on the image data of one or more colonies detected by preprocessor 222, analyzing the encoded image to detect the at least one color of the organism on the nutrient media in container 214. More specifically, once preprocessor 222 defines (by colony detection and intensity threshold) the interested area for color detection, the pixels are collected and classified into n number of bins. The process is generally commenced with an arbitrary n value. Thereafter the bins are merged after each step (say, if two bin fall within the profile of the same species). Since colonies on a plate may contain a mixed culture of different organisms, classification is utilized to better represent the color of colonies on a plate. Profile colors for different species are established. Each color profile has an interval which defines the color range, e.g. Profile A is defined as a spherical area centered around Color A. The classification process starts with an arbitrary value n and divides the all colony area pixels into n bins. The average color of each bin is calculated as Color Bi (i=1 to n). If any two of the Color Bi falls within the same spherical area, they are merged and a new Color Bi is given. All colony area pixels are then reclassified into the remaining bins and a new set of the average colors is calculated. This process is repeated until the classification is stable. In the end, one or more bins are left as the representative color of the colonies detected.

Edge detector 226, together with an edge counter 228, is adapted to measure a non-chromatic optical characteristic of the organism on the nutrient media, in particular, the texture of the organism's colonies. Edge detector 226 operates on the image data of one or more colonies detected by preprocessor 222, analyzing the image data to detect edges within any given colony. Edge counter 228 then tallies the edges detected within a predetermined unit area of the respective colony.

Color processor 224 and edge counter 228 are connected at respective outputs to a memory 230 that stores the chromatic and textural measurements pertaining to the unknown specimen of container 214. Memory 230 may store several color measurements and several textural measurements for each specimen and possibly for each colony in container 214. Alternatively, color processor 224 and edge counter 228 may be programmed to generate an average measurement over one or more colonies in the specimen container 214.

Computer 212 includes a comparator module 232 connected to memory 230 and to library 208 for comparing the color and texture measurements in memory 230 with chromatic and non-chromatic optical characteristics of known organisms stored in library 208. Comparator module 232 supplies the results of its operations to an output peripheral 234 such as a computer monitor, a printer, an Internet connection, etc.

Electronic library 208 stores chromatic characteristics (HSI values) and associated non-chromatic characteristics such as texture (e.g., edge counts) and, optionally, characteristic colony size (per predetermined growth time) of a multiplicity of known organisms. The color, texture, and size characteristics of the known organisms are each preferably in the form of at least one ordered pair including an average value and a statistical measure of variation about the average value, such as a predetermined number of standard deviations. With respect to color, library 208 stores, for each known organism, an average hue, an average saturation, and an average intensity, together with respective deviation values. With respect to texture, library 208 stores for each known organism an average edge count per unit area and a measure of deviation about that aver edge count. With respect to size, library 208 stores an average radius, area, volume or mass, as well as a respective deviation value. Alternatively viewed, library 208 stores a distribution as a center value and an interval radius with certain confidence (say 90%). Comparator 232 calculates the probability of the unknown sample in each of the profile distributions. The higher the probability, the more likely the sample would match the profile of a certain species.

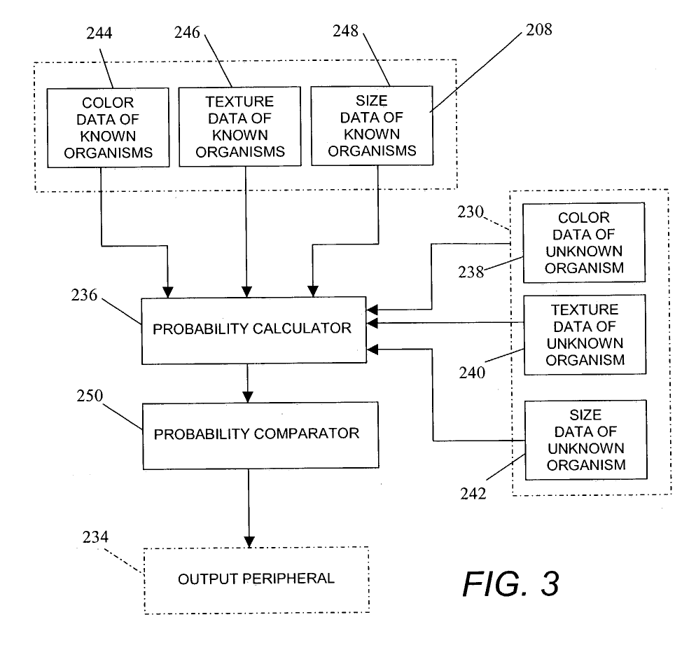

As depicted in FIG. 3, comparator module 232 includes a probability calculator 236 for determining, for each given one of a plurality of known organisms with pre-identified chromatic characteristics and pre-identified non-chromatic optical characteristics stored in encoded form in library 208, a probability that the unknown organism in specimen container 214 is of the same type as the given one of the known organisms. Probability calculator 236 is operatively connected to color processor 224 and edge counter 228 via memory 230. Probability calculator 236 receives color data 238, texture data 240, and size data 242 from memory 230 pertaining to the unknown organism in container 214. In addition, probability calculator 236 accesses a color data memory bank 244, a texture data memory area 246, and a size data store 248 in library 208. Generally, probability calculator 236 undertakes its computations on the basis of all the types of data available. However, it is possible for a probability computation to be limited, for example, to color data.

More specifically, each of the image features is an independent axis in a multi-dimension space. With color, texture and size, the space would be 3-D. Since the color itself if actually 3-D (HSV or HSI), the resulting space is 5-D. Since these features do not share a common definition, probability is used to evaluate the distance between the unknown sample and known profile. For example, species A and species B are known species with respective profiles, whereas an unknown sample has color a, size b and texture c. Calculator 236 computes a set of probability values p(A,a), p(B,a), p(C,a), p(A,b), p(B,b), p(C,b), p(A,c), p(B,c), p(C,c). On the assumption that these features are independent behaviors of the sample space, it is possible to calculate the combined probability values simply by multiplying them together. If p(h)=p(A,a)p(A,b)p(A,c) and p(g)=p(B,a)p(B,b)(pB,c), p(k)=p(C,a)p(C,b)p(C,c), then p(A|a,b,c)=p(h)/(p(h)+p(g)+p(k)), p(B|a,b,c)=p(g)/(p(h)+p(g)+p(k)), p(C|a,b,c)=p(k)/(p(h)+p(g)+p(k)).

Probability calculator 236 feeds its probability determinations to a comparator 250 programmed to determine the type of known organism having the highest calculated probability for the unknown specimen in container 214. The outcome of that determination is communicated to the user via output peripheral 234. Comparator 250 may be programmed to communicate several possible organism identities where the calculated probabilities are close to one another. The calculated probability is generally provided to the user together with an identification of the possibly matching organism.

It is to be noted that the apparatus of FIGS. 1-3 may be used to identify unknown organisms also where the chromagenic phenomenon includes more than one color, for instance, where a colony of the organism has a central region characterized by a first color (HSI) and texture and a surrounding region or halo characterized by a second color (HSI) and texture. In that case, preprocessor 222 and/or color processor 224 and edge detector 226 detect the existence of the multiple regions and distinguish the inner region from the outer region. Comparator 232 and probability calculator 236 need calculate probabilities only for those known organisms exhibiting a halo.

It is to be further noted that the color information is taken by sampling the colony areas with a median illumination intensity (the color information is not reliable when intensity is too low or high). For each species, the profile stored in library 208 (or 118) specifies a range of intensity where the majority of the individual occurrence would lie (e.g., 99% of the colonies have an intensity of 20-50% gray). The color information is extracted only for colonies falling within that intensity range. However, if the intensity in the profile is somewhat high or low (close to 0 or 100%), then color information is not extracted above or below a pre-determined range (ex. 10-90%) and the image area is considered either under or over exposed.

Techniques such as classification may be used to get better color representation of the colonies detected. If more than one color representation is required, the plate 214 may contain mixed culture. The user may specify a specific colony (e.g., via a mouse click on an image shown on peripheral 234) for extraction of a colony with better representation.

The probability of likeness of the unknown colony(s) to a profile in library 118 or 208 may be also calculated and combined with a certain weighting factor to produce the final result. Once the matching is done, the unknown sample may be added to the profile library 118 or 208.

The apparatus of FIG. 2 may be used to not only identify an organism but to simultaneously determine susceptibility of the organism to one or more antibiotics. In that event, the nutrient media in container 214 is provided with one or more antibiotic delivery vehicles such as biocide-impregnated disks. To determine susceptibility of the specimen organism to one or more antibiotics, computer 212 includes a susceptibility calculator module 252 (FIG. 2). The operation of that module, in cooperation with preprocessor 222, is described in U.S. Pat. Nos. 4,701,850, 6,107,054, and 6,238,879, the disclosures of which are hereby incorporated by reference herein. Basically, susceptibility calculator module 252 measures the diameters, radii or areas of microbe inhibition zones about the antibiotic disks and determines minimum inhibitory concentrations therefrom.

This combination of organism identification and susceptibility determination may be performed, for instance, where the unknown organism in container 214 is a mold. In one procedure, the nutrient media in container 214 is provided with at least one anti-fungal composition, and pieces of the mold are deposited in an array on the nutrient media provided with the anti-fungal composition. The mold is then grown on the nutrient media. In this case, susceptibility calculator module 252 functions in part as a size detector for determining a size parameter of mold grown from at least one of the pieces to thereby ascertain the susceptibility of the mold to the anti-fungal composition.

It is to be noted that yeast and bacteria form individual colonies growing on the agar surface. These colonies may grow together to appear like confluent growth, but the preset method calls for inoculating plates based on “cfu” (colony forming units/individual cells) to produce after incubation a non-confluent or just barely confluent lawn of organism growth. Molds form a single matt of inlocking-hyphael-growth (multicellular interwoven).

Because yeast and bacteria produce individual-colonies that may have unique genetic profiles expressed in this assay as “some resistant colonies, or colonies with varying resistant-susceptibility patterns”. Molds produce a confluent lawn of a multicellular organism, that has a relatively uniform drug resistant-susceptibility profile to the drug(s) tested. Therefore, although reading molds is more difficult in some ways, it is also simpler in that it is unnecessary to look for drug-resistant colonies/organisms growing inside the zone-of-growth-inhibition.

With regard to simultaneous identification and susceptibility testing, some molds require user input of microscopic observations as to morphology to narrow the domain of possible species to a group before reading the plate 214. This is usual in all microbiology, as bacteria are always categorized as gram-negative rod, gram negative cocci, gram positive rod, gram positive cocci, before proceding with futher ID and AST tests. With molds, the user may need to examine the spores produced by the mold, before reading, as spores are the most important microscopic feature of the mold.

To repeat, molds are basically identified to species only using morphology, that is, macroscopic appearance (appearance on the agar plate) and sometimes microscopic appearance (spore morphology). This is unique in itself, as bacteria and yeast require biochemical testing to identify to the species level, morphology being only tentative, as these single cell organisms all look more or less alike. This unique feature of molds enabled the present unique approach of camera reading (only) to do both ID and AST simultaneously (without biochemical tests).

As discussed above, the apparatus of FIG. 2 may be used in a microbiological-assay method for testing an organism grown on solid nutrient media for antibiotic susceptibility. In a particular example of such an antibiotic testing procedure, an elongate strip provided at different locations with different concentrations of an antibiotic composition is disposed in container 214 together with an organism of unknown type. After a predetermined incubation period, camera or CCD 204 scans the nutrient media, the strip, and a growth region of the organism. Camera or CCD 204 generates an analog or digital electrical signal encoding an image of the strip and the growth region on the nutrient media in container 204. The encoded image is stored in digital format in memory 206, as discussed above, and is digitally processed by preprocessor 222 to detect the antibiotic strip and a boundary or edge of the growth region on the nutrient media. Antibiotic susceptibility calculator 252 then operates to determine an intersection point of an edge of the growth region and the antibiotic strip to thereby determine a minimum inhibitory concentration of the antibiotic composition based on the detected intersection point. Antibiotic susceptibility calculator 252 may be programmed to read graduation marks along the test strip to thereby read the minimum inhibitory concentration.

As an aid to the identification process described herein, and/or for purposes of enabling a more specific identification, ancillary information derived from sources other than the camera 204 may be furnished to computer 212. For instance, information derived from microscope observations may be manually input and compared by computer 212 with data stored in the electronic library 208. The observations may be a verbal description of structural characteristics of the subject organism, as opposed to textural characteristics. In the case of a cellular organism, the observations might entail a characterization of the nucleus or the mitochondria. For a mold, the observations might include a qualitative characterization of colony shape or growth patterns.

Alternatively or additionally, an operator or lab technician may input into computer 212 growth rate and/or incubation duration. The operating of computer 212 then includes comparing the input growth rate and/or incubation duration with corresponding data stored in the library.

An input peripheral such as a keyboard (not shown) is operatively connected to memory 230 or comparator 232 for enabling a comparison of additional information such as microscopic characteristics, growth rate, or incubation duration with corresponding data in library 208. With respect to the latter, for one or more known organisms, library 208 may be loaded with calorimetric, size, and texture data that may vary in accordance with the incubation duration.

It is to be noted that the image information preferably includes the color of the semisolid nutrient media surrounding the imaged colony or colonies. This ambient color information may be used for calibration purposes, to fine tune the color detection process, for identification of the nutrient medium, etc.

In a particular embodiment of a method utilizing the apparatus of FIGS. 1-3, an organism is identified based on only one image of one plate carrying chromogenic media. Computer 212, and more particularly, preprocessor 222 and edge detector 226, is programmed to recognize that there are multiple colonies on the semisolid nutrient media of container 214 and to process the visible characteristics of those colonies separately to make identifications of several organisms substantially simultaneously. This technique is a substantial improvement over current techniques that utilize a different agar plate for each identification and is useful in water-sample tests for E. coli and other bacteria. This technique enables identification to proceed from the primary or initial agar media plate on which a specimen was first grown.

As described above, an antibiotic susceptibility disk test may be read simultaneously on the same nutrient media as the identification is performed, at least as far as some organisms are concerned. This procedure may require additional input into computer 212, for instance, pertaining to microscopic observations, and or growth-rate/incubation time. One example of this is in the identification and disk susceptibility testing of fungal molds.

With some organisms, a count of the colonies of organism(s) (e.g., by preprocessor 222 and edge detector 226) may be performed simultaneously on the same nutrient media as the identification. Some of these organisms may require additional input of microscopic observations. One example of this is water-sample tests for E. coli and other bacteria.

Although the invention has been described in terms of particular embodiments and applications, one of ordinary skill in the art, in light of this teaching, can generate additional embodiments and modifications without departing from the spirit of or exceeding the scope of the claimed invention. For instance, where computer 212 is connected to microscope (not shown) fitted with a charge-coupled device or other optical sensor, computer 212 may be programmed to automatically identify microscopic structural characteristics such as spore formation and shape or staining characteristics as color. The parameters could include shape, size, and internal texture. Comparisons with prerecorded information pertaining to known species could be performed automatically as described hereinabove with reference to more macroscopic characteristics. Accordingly, it is to be understood that the drawings and descriptions herein are proffered by way of example to facilitate comprehension of the invention and should not be construed to limit the scope thereof.

What is claimed is:

1. A microbiological-assay method for identifying an organism grown on one chromagenic semisolid nutrient media, said organism exhibiting at least one color on said nutrient media, said method comprising:
generating a digitized electrical signal encoding an image of said organism on said nutrient media;
storing the encoded image;
digitally processing said encoded image to detect the at least one color of said organism on said nutrient media;
storing chromatic characteristics of a multiplicity of known organisms in an electronic library; and
operating a computer to compare the detected color of said organism with chromatic characteristics stored in said library.

2. The method defined in claim 1 wherein the operating of said computer includes calculating, for each given one of a plurality of known organisms with pre-identified chromatic characteristics stored in encoded form in said library, a probability that said organism is of the same type as said given one of said known organisms.

3. The method defined in claim 2 wherein the chromatic characteristics stored in said library include, for each of said plurality of known organisms, a plurality of chromatic parameters.

4. The method defined in claim 3 wherein said chromatic parameters include at least one characteristic hue, at least one characteristic saturation, and at least one characteristic value or intensity.

5. The method defined in claim 3 wherein said chromatic parameters each include an average value and a statistical measure of variation about said average value.

6. The method defined in claim 1, further comprising preliminarily processing said encoded image to detect colonies of said organism on said nutrient media, the processing of said encoded image to detect the at least one color of said organism being performed with reference to image data pertaining to at least a selected one of the detected colonies.

7. The method defined in claim 6 wherein the preliminary processing of said encoded image includes digitally measuring at least one parameter distinguishing said colonies from said nutrient media.

8. The method defined in claim 7 wherein said at least one parameter is light intensity.

9. The method defined in claim 6 wherein the preliminary processing of said encoded image includes automatically counting the colonies, whereby a colony count is performed simultaneously on the same nutrient media as the organism identification.

10. The method defined in claim 6 wherein the processing of said encoded image includes detecting a plurality of colors each associated with a respective one of the colonies detected on said nutrient media, the operating of said computer including comparing the detected colors with chromatic characteristics stored in said library, to identify multiple organisms grown on said nutrient media.

11. The method defined in claim 1, further comprising storing in said library at least one non-chromatic optical characteristic of each of said known organisms, the digital processing of said encoded image including measuring the at least one non-chromatic optical characteristic of said organism on said nutrient media, the operating of said computer including comparing the measured non-chromatic optical characteristic of said organism with the non-chromatic optical characteristics stored in said library.

12. The method defined in claim 11 wherein said at least one non-chromatic optical characteristic is a textural characteristic.

13. The method defined in claim 12 wherein the measuring of said textural characteristic includes detecting edges in said encoded image and counting detected edges per unit area.

14. The method defined in claim 11 wherein the operating of said computer includes calculating, for each given one of a plurality of known organisms with pre-identified chromatic characteristics and pre-identified non-chromatic optical characteristics stored in encoded form in said library, a probability that said organism is of the same type as said given one of said known organisms.

15. The method defined in claim 14 wherein said chromatic characteristics and said non-chromatic optical characteristics each include an average value and a statistical measure of variation about said average value.

16. The method defined in claim 1 wherein the generating of said digitized electrical signal includes scanning said media and said organism with an optical scanner.

17. The method defined in claim 16 wherein said optical scanner is taken from the group consisting of a camera, a digital camera, and a charge-coupled device.

18. The method defined in claim 1 wherein said organism is taken from the group consisting of yeast, bacteria, and mold.

19. The method defined in claim 1 wherein said organism is a mold, further comprising:
providing said nutrient media with at least one anti-fungal composition;
depositing pieces of said mold in an array on said nutrient media provided with said anti-fungal composition;
growing said mold on said nutrient media provided with said anti-fungal composition; and
measuring effectiveness of said anti-fungal composition, the measuring of effectiveness including operating said computer to determine a size parameter of mold grown from at least one of said pieces.

20. The method defined in claim 19, further comprising inputting into said computer additional information derived from microscope observations, the operating of said computer including comparing said additional information with data stored in said library.

21. The method defined in claim 19, further comprising inputting into said computer additional information taken from the group consisting of growth rate and incubation duration, the operating of said computer including comparing said additional information with data stored in said library.

22. The method defined in claim 1, further comprising operating said computer to automatically determine an antibiotic susceptibility of said organism on said nutrient media, whereby antibiotic susceptibility and organism identification are determined simultaneously from the same plate of inoculated nutrient media.

23. The method defined in claim 22 wherein the digital processing of said encoded image includes measuring a growth-inhibition zone on said nutrient media, the operating of said computer to automatically determine the antibiotic susceptibility of said organism including determining a minimum inhibitory concentration of an antibioitic from the measurement of the growth-inhibition zone.

24. The method defined in claim 1, further comprising inputting into said computer additional information derived from microscope observations, the operating of said computer including comparing said additional information with data stored in said library.

25. The method defined in claim 1, further comprising inputting into said computer additional information taken from the group consisting of growth rate and incubation duration, the operating of said computer including comparing said additional information with data stored in said library.

26. The method defined in claim 1 wherein the processing of said encoded image includes detecting a plurality of colors each associated with a respective colony on said nutrient media, the operating of said computer including comparing the detected colors with chromatic characteristics stored in said library, to identify multiple organisms grown on said nutrient media.

27. The method defined in claim 1 wherein said nutrient media is the primary or initial agar media plate on which a patient's specimen was first grown.

28. A microbiological-assay apparatus comprising: a support for holding a container of chromagenic semisolid nutrient media wherein an organism of unknown identity is grown, said organism exhibiting at least one color on said nutrient media;
an optical scanning device aimed at said support for generating a digitized electrical signal encoding an image of said organism on said nutrient media;
a memory operatively connected to said scanning device for temporarily storing the encoded image;
a digital processor operatively connected to said memory for analyzing said encoded image to detect the at least one color of said organism on said nutrient media;
an electronic library storing chromatic characteristics of a multiplicity of known organisms; and
a computer operatively connected to said processor and said library, said computer being programmed to compare the detected color of said organism with chromatic characteristics stored in said library.

29. The apparatus defined in claim 28 wherein said computer is programmed to calculate, for each given one of a plurality of known organisms with pre-identified chromatic characteristics stored in encoded form in said library, a probability that said organism is of the same type as said given one of said known organisms.

30. The apparatus defined in claim 29 wherein the chromatic characteristics stored in said library include, for each of said plurality of known organisms, a plurality of chromatic parameters.

31. The apparatus defined in claim 30 wherein said chromatic parameters include at least one characteristic hue, at least one characteristic saturation, and at least one characteristic value or intensity.

32. The apparatus defined in claim 30 wherein said chromatic parameters each include an average value and a statistical measure of variation about said average value.

33. The apparatus defined in claim 28, further comprising a preprocessor operatively connected to said memory for preliminarily processing said encoded image to detect colonies of said organism on said nutrient media, said processor being operatively connected to said preprocessor to operate on image data pertaining to a selected one of the detected colonies.

34. The apparatus defined in claim 33 wherein said preprocessor includes a module for digitally measuring at least a light intensity parameter.

35. The apparatus defined in claim 28 wherein said library stores at least one non-chromatic optical characteristic of each of said known organisms, said processor including means for measuring the at least one non-chromatic optical characteristic of said organism on said nutrient media, said computer including a comparator module for comparing the measured non-chromatic optical characteristic of said organism with the non-chromatic optical characteristics stored in said library.

36. The apparatus defined in claim 35 wherein said at least one non-chromatic optical characteristic is a textural characteristic.

37. The apparatus defined in claim 36 wherein said processor includes an edge detector and an edge counter.

38. The apparatus defined in claim 35 wherein said computer includes a probability calculator for determining, for each given one of a plurality of known organisms with pre-identified chromatic characteristics and pre-identified non-chromatic optical characteristics stored in encoded form in said library, a probability that said organism is of the same type as said given one of said known organisms.

39. The apparatus defined in claim 38 wherein said chromatic characteristics and said non-chromatic optical characteristics each include an average value and a statistical measure of variation about said average value.

40. The apparatus defined in claim 28 wherein said organism is a mold, said nutrient media being provided with at least one anti-fungal composition, pieces of said mold being deposited in an array on said nutrient media provided with said anti-fungal composition, said mold being grown on said nutrient media provided with said anti-fungal composition, said computer including size detector for determining a size parameter of mold grown from at least one of said pieces.

41. The apparatus defined in claim 28 wherein said processor and said computer comprise program-modified generic digital circuits of the same electronic machine.

42. A microbiological-assay method for testing an organism grown on solid nutrient media for antibiotic susceptibility, comprising:
providing a container of semisolid nutrient media on which is disposed an organism of unknown type and an elongate strip provided at different locations with different concentrations of an antibiotic composition;
after an incubation period, optically scanning the nutrient media, said strip, and a growth region of said organism;
in response to the optical scanning, generating a digitized electrical signal encoding an image of said strip and said growth region on said nutrient media;
storing the encoded image;
digitally processing said encoded image to detect an intersection point of an edge of said growth region and said strip; and
operating a computer to determine a minimum inhibitory concentration of said antibiotic composition based on the detected intersection point.

43. The method defined in claim 42 wherein the processing of said encoded image includes operating said computer.
